(19) United States
(12) Patent Application Publication
(10) Pub. No.: US 2020/0324117 A1
(43) Pub. Date: Oct. 15, 2020

(54) SYSTEMS AND METHODS OF GENERATING STIMULATION PATTERNS

(71) Applicant: ADVANCED NEUROMODULATION SYSTEMS, INC., Plano, TX (US)

(72) Inventors: Simeng ZHANG, Frisco, TX (US); Alexander Kent, Mountain View, CA (US)

(21) Appl. No.: 16/384,528

(22) Filed: Apr. 15, 2019

(51) Int. Cl. A61N 1/36; A61N 1/05; G06N 5/04

(52) U.S. Cl. CPC A61N 1/36128; A61N 1/0534; G06N 5/043; A61N 1/36067; A61N 1/0551

(57) ABSTRACT

The present disclosure provides systems and methods for generating stimulation patterns. A computing device includes a processor, and a memory device communicatively coupled to the processor. The memory device includes instructions that, when executed, cause the processor to provide a plurality of inputs to a multi-objective modified binary particle swarm optimization (MOMBPSO) algorithm, and apply the MOMBPSO to a computational circuit model using the plurality of inputs to generate a plurality of candidate stimulation patterns, wherein the MOMBPSO is applied to the computational circuit model to optimize both i) therapy efficacy and ii) power utilization.

### A. FIELD OF THE DISCLOSURE

The present disclosure relates generally to neurostimulation systems, and more particularly to generating stimulation patterns using multi-objective modified binary particle swarm optimization (MOMBPSO).

### B. BACKGROUND ART

Deep brain stimulation (DBS) is an established neuromodulation therapy for the treatment of movement disorders and has been shown to improve cardinal motor symptoms of Parkinson's Disease (PD), such as bradykinesia, rigidity, and tremors. These improvements generally occur within a few minutes of initiation of stimulation, and disappear within a similar timeframe after stimulation is discontinued.

Traditional DBS stimulation patterns may use a single tonic frequency (e.g., at 100 or 130 Hertz (Hz)). Although tonic stimulation patterns are effective at treating the symptoms of movement disorders such as PD or essential tremor, there may be other stimulation patterns that would provide better (or equal therapy) while having a smaller power utilization. For example, it has been found that an irregular waveform with a spontaneous frequency of 45 Hz performs equally as well as tonic DBS at higher frequencies in both preclinical and clinical settings. However, at least some techniques for identifying non-tonic stimulation patterns focus on only a single objective (e,g., DBS therapy efficacy). Accordingly, it would be desirable to identify new stimulation patterns while satisfying multiple objectives simultaneously.

### BRIEF SUMMARY OF THE DISCLOSURE

In one embodiment, the present disclosure is directed to a computing device for generating stimulation patterns for neurostimulation. The computing device includes a processor, and a memory device communicatively coupled to the processor. The memory device includes instructions that, when executed, cause the processor to provide a plurality of inputs to a multi-objective modified binary particle swarm optimization (MOMBPSO) algorithm, and apply the MOMBPSO to a computational circuit model using the plurality of inputs to generate a plurality of candidate stimulation patterns, wherein the MOMBPSO is applied to the computational circuit model to optimize both i) therapy efficacy and ii) power utilization.

In another embodiment, the present disclosure is directed to a computer-implemented method of generating stimulation patterns for neurostimulation. The method includes providing, using a processor, a plurality of inputs to a multi-objective modified binary particle swarm optimization (MOMBPSO) algorithm, and applying, using the processor, the MOMBPSO to a computational circuit model using the plurality of inputs to generate a plurality of candidate stimulation patterns, wherein the MOMBPSO is applied to the computational circuit model to optimize both i) therapy efficacy and ii) power utilization.

The foregoing and other aspects, features, details, utilities and advantages of the present disclosure will be apparent from reading the following description and claims, and from reviewing the accompanying drawings.

### BRIEF DESCRIPTION OF THE DRAWINGS

FIG. 1 is a schematic view of one embodiment of a stimulation system.

FIG. 2 is a block diagram of one embodiment of a computing device that may be used to generate stimulation patterns.

FIG. 3 is a schematic diagram illustrating an error index.

FIG. 4 is a schematic diagram of the Rubin-Terman model.

FIGS. 5A and 5B are a flow diagram of one embodiment of a method for generating stimulation patterns.

FIG. 6 is a graph showing multiple final estimated Pareto fronts generated using the method shown in FIGS. 5A and 5B.

FIGS. 7A-7F are graphs showing six candidate stimulation patterns generated using the method shown in FIGS. 5A and 5B.

### DETAILED DESCRIPTION OF THE DISCLOSURE

The present disclosure provides systems and methods for generating stimulation patterns (e.g., for deep brain stimulation (DBS)) using multi-objective modified binary particle swarm optimization (MOMBPSO). Using MOMBPSO, multiple objectives (e.g., DBS therapy efficiency and power utilization) of optimization are achievable. Further, MOMBSPSO may be coupled with the Rubin-Terman model of the basal ganglia to generate new, improved stimulation patterns. For example, the systems and methods described herein may be used to generate stimulation patterns that are as effective as 100+ Hz tonic stimulation, but with lower power utilization, or stimulation patterns that are more effective than 100+ Hz tonic stimulation, with equivalent power utilization.

Neurostimulation systems are devices that generate electrical pulses and deliver the pulses to nerve tissue of a patient to treat a variety of disorders. One category of neurostirnulation systems is DBS. In OBS, electrical pulses are delivered to parts of a subject's brain, for example, for the treatment of movement and effective disorders such as PD and essential tremor.

Neurostimulation systems generally include a pulse generator and one or more leads. A stimulation lead includes a lead body of insulative material that encloses wire conductors. The distal end of the stimulation lead includes multiple electrodes, or contacts, that are electrically coupled to the wire conductors. The proximal end of the lead body includes multiple terminals (also electrically coupled to the wire conductors) that are adapted to receive electrical pulses. In DBS systems, the stimulation lead is implanted within the brain tissue to deliver the electrical pulses. The stimulation leads are then tunneled to another location within the patient's body to be electrically connected with a pulse generator or, alternatively, to an “extension.” The pulse generator is typically implanted within a subcutaneous pocket created during the implantation procedure.

The pulse generator is typically implemented using a metallic housing that encloses circuitry for generating the electrical pulses, control circuitry, communication circuitry, a rechargeable battery, etc. The pulse generating circuitry is coupled to one or more stimulation leads through electrical connections provided in a “header” of the pulse generator. Specifically, feedthrough wires typically exit the metallic housing and enter into a header structure of a moldable material. Within the header structure, the feedthrough wires are electrically coupled to annular electrical connectors. The header structure holds the annular connectors in a fixed arrangement that corresponds to the arrangement of terminals on a stimulation lead.

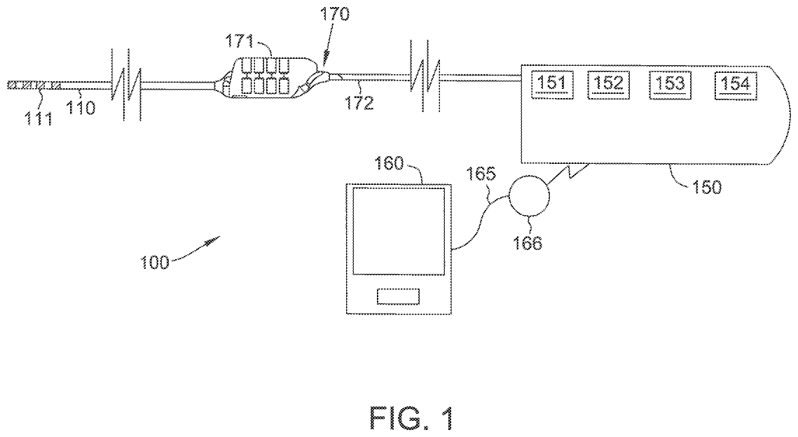

Referring now to the drawings, and in particular to FIG. 1, a stimulation system is indicated generally at 100. Stimulation system 100 generates electrical pulses for application to tissue of a patient, or subject, according to one embodiment. System 100 includes an implantable pulse generator (IPG) 150 that is adapted to generate electrical pulses for application to tissue of a patient. IPG 150 typically includes a metallic housing that encloses a controller 151, pulse generating circuitry 152, a battery 153, far-field and/or near field communication circuitry 154, and other appropriate circuitry and components of the device. Controller 151 typically includes a microcontroller or other suitable processor for controlling the various other components of the device. Software code is typically stored in memory of IPG 150 for execution by the microcontroller or processor to control the various components of the device.

IPG 150 may comprise one or more attached extension components 170 or be connected to one or more separate extension components 170. Alternatively, one or more stimulation leads 110 may be connected directly to IPG 150. Within IPG 150, electrical pulses are generated by pulse generating circuitry 152 and are provided to switching circuitry. The switching circuit connects to output wires, traces, lines, or the like (not shown) which are, in turn, electrically coupled to internal conductive wires (not shown) of a lead body 172 of extension component 170. The conductive wires, in turn, are electrically coupled to electrical connectors (e.g., “Bal-Seal” connectors) within connector portion 171 of extension component 170. The terminals of one or more stimulation leads 110 are inserted within connector portion 171 for electrical connection with respective connectors. Thereby, the pulses originating from IPG 150 and conducted through the conductors of lead body 172 are provided to stimulation lead 110. The pulses are then conducted through the conductors of lead 110 and applied to tissue of a patient via electrodes 111. Any suitable known or later developed design may be employed for connector portion 171.

For implementation of the components within IPG 150, a processor and associated charge control circuitry for an implantable pulse generator is described in U.S. Pat. No. 7,571,007, entitled “SYSTEMS AND METHODS FOR USE IN PULSE GENERATION,” which is incorporated herein by reference. Circuitry for recharging a rechargeable battery of an implantable pulse generator using inductive coupling and external charging circuits are described in U.S. Pat. No. 7,212,110, entitled “IMPLANTABLE DEVICE AND SYSTEM FOR WIRELESS COMMUNICATION,” which is incorporated herein by reference.

An example and discussion of “constant current” pulse generating circuitry is provided in U.S. Patent Publication No. 2006/0170486 entitled “PULSE GENERATOR HAVING AN EFFICIENT FRACTIONAL VOLTAGE CONVERTER AND METHOD OF USE,” which is incorporated herein by reference. One or multiple sets of such circuitry may be provided within IPG 150. Different pulses on different electrodes may be generated using a single set of pulse generating circuitry using consecutively generated pulses according to a “multi-stim set program” as is known in the art. Alternatively, multiple sets of such circuitry may be employed to provide pulse patterns that include simultaneously generated and delivered stimulation pulses through various electrodes of one or more stimulation leads as is also known in the art. Various sets of parameters may define the pulse characteristics and pulse timing for the pulses applied to various electrodes as is known in the art. Although constant current pule generating circuitry is contemplated for some embodiments, any other suitable type of pulse generating circuitry may be employed such as constant voltage pulse generating circuitry.

Stimulation lead(s) 110 may include a lead body of insulative material about a plurality of conductors within the material that extend from a proximal end of lead 110 to its distal end. The conductors electrically couple a plurality of electrodes 111 to a plurality of terminals (not shown) of lead 110. The terminals are adapted to receive electrical pulses and the electrodes 111 are adapted to apply stimulation pulses to tissue of the patient. Also sensing of physiological signals may occur through electrodes 111, the conductors, and the terminals. Additionally or alternatively, various sensors (not shown) may be located near the distal end of stimulation lead 110 and electrically coupled to terminals through conductors within the lead body 172. Stimulation lead 110 may include any suitable number and type of electrodes 111, terminals, and internal conductors.

Controller device 160 may be implemented to recharge battery 153 of IPG 150 (although a separate recharging device could alternatively be employed). A “wand” 165 may be electrically connected to controller device through suitable electrical connectors (not shown). The electrical connectors are electrically connected to coil 166 (the “primary” coil) at the distal end of wand 165 through respective wires (not shown). Typically, coil 166 is connected to the wires through capacitors (not shown). Also, in some embodiments, wand 165 may comprise one or more temperature sensors for use during charging operations.

The patient then places the primary coil 166 against the patient's body immediately above the secondary coil (not shown), i.e., the coil of the implantable medical device. Preferably, the primary coil 166 and the secondary coil are aligned in a coaxial manner by the patient for efficiency of the coupling between the primary and secondary coils. Controller device 160 generates an AC-signal to drive current through coil 166 of wand 165. Assuming that primary coil 166 and secondary coil are suitably positioned relative to each other, the secondary coil is disposed within the field generated by the current driven through primary coil 166. Current is then induced in secondary coil. The current induced in the coil of the implantable pulse generator is rectified and regulated to recharge battery of IPG 150. The charging circuitry may also communicate status messages to controller device 160 during charging operations using pulse-loading or any other suitable technique. For example, controller device 160 may communicate the coupling status, charging status, charge completion status, etc.

External controller device 160 is also a device that permits the operations of IPG 150 to be controlled by user after IPG 150 is implanted within a patient, although in alternative embodiments separate devices are employed for charging and programming. Also, multiple controller devices may be provided for different types of users (e,g., the patient or a clinician). Controller device 160 can be implemented by utilizing a suitable handheld processor-based system that possesses wireless communication capabilities. Software is typically stored in memory of controller device 160 to control the various operations of controller device 160. Also, the wireless communication functionality of controller device 160 can be integrated within the handheld device package or provided as a separate attachable device. The interface functionality of controller device 160 is implemented using suitable software code for interacting with the user and using the wireless communication capabilities to conduct communications with IPG 150.

Controller device 160 preferably provides one or more user interfaces to allow the user to operate IPG 150 according to one or more stimulation programs to treat the patient's disorder(s). Each stimulation program may include one or more sets of stimulation parameters including pulse amplitude, pulse width, pulse frequency or inter-pulse period, pulse repetition parameter (e.g., number of times for a given pulse to be repeated for respective stim set during execution of program), etc. In the methods and systems described herein, parameters may include, for example, a number of pulses in a burst (e.g., 3, 4, or 5 pulses per burst), an intra-burst frequency (e,g., 130 Hz), an inter-burst frequency (e.g., 3-20 Hz), and a delay between a first and second burst (e.g., less than 1 millisecond (ms)).

IPG 150 modifies its internal parameters in response to the control signals from controller device 160 to vary the stimulation characteristics of stimulation pulses transmitted through stimulation lead 110 to the tissue of the patient. Neurostimulation systems, stim sets, and multi-stim set programs are discussed in PCT Publication No. WO 2001/093953, entitled “NEUROMODULATION THERAPY SYSTEM,” and U.S. Pat. No. 7,228,179, entitled “METHOD AND APPARATUS FOR PROVIDING COMPLEX TISSUE STIMULATION PATTERNS,” which are incorporated herein by reference. Example commercially available neurostimulation systems include the EON MINI™ pulse generator and RAPID PROGRAMMER™ device from Abbott Laboratories.

As described above, the systems and methods described herein relate to using multi-objective modified binary particle swarm (MOMBPSO) in combination with the Rubin-Terman model (RT model) of the basal ganglia to generate improved stimulation patterns for DBS. The stimulation patterns may target, for example, the subthalamic nucleus (STN), the globus pallidus interna (GPi), or the globus pallidus externa (GPe) of the brain.

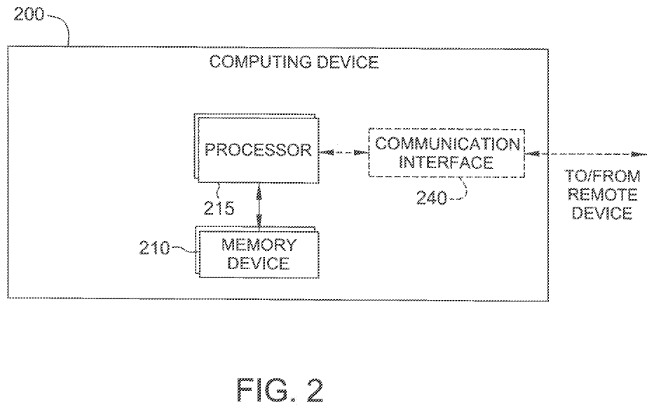

FIG. 2 is a block diagram of one embodiment of a computing device 200 that may be used to generate stimulation patterns as described herein. Computing device 200 may be included, for example, within an IPG (e.g., IPG 150) or an external pulse generator.

In this embodiment, computing device 200 includes at least one memory device 210 and a processor 215 that is coupled to memory device 210 for executing instructions. In some embodiments, executable instructions are stored in memory device 210. In the illustrated embodiment, computing device 200 performs one or more operations described herein by programming processor 215. For example, processor 215 may be programmed by encoding an operation as one or more executable instructions and by providing the executable instructions in memory device 210.

Processor 215 may include one or more processing units (e.g., in a multi-core configuration). Further, processor 215 may be implemented using one or more heterogeneous processor systems in which a main processor is present with secondary processors on a single chip. In another illustrative example, processor 215 may be a symmetric multi-processor system containing multiple processors of the same type. Further, processor 215 may be implemented using any suitable programmable circuit including one or more systems and microcontrollers, microprocessors, reduced instruction set circuits (RISC), application specific integrated circuits (ASIC), programmable logic circuits, field programmable gate arrays (FPGA), and any other circuit capable of executing the functions described herein.

In the illustrated embodiment, memory device 210 is one or more devices that enable information such as executable instructions and/or other data to be stored and retrieved. Memory device 210 may include one or more computer readable media, such as, without limitation, dynamic random access memory (DRAM), read-only memory (ROM), electrically erasable programmable read-only memory (EEPROM), static random access memory (SRAM), a solid state disk, and/or a hard disk. Memory device 210 may be configured to store, without limitation, application source code, application object code, source code portions of interest, object code portions of interest, configuration data, execution events and/or any other type of data.

Computing device 200, in the illustrated embodiment, includes a communication interface 240 coupled to processor 215. Communication interface 240 communicates with one or more remote devices, such as a clinician or patient programmer. To communicate with remote devices, communication interface 240 may include, for example, a wired network adapter, a wireless network adapter, a radio-frequency (RF) adapter, and/or a mobile telecommunications adapter.

MOMBPSO allows for optimizing multiple objectives (e.g., therapy efficacy and power utilization), as opposed to modified binary particle swarm optimization (MBPSO), which optimizes a single objective. MBPSO is described in “A novel binary particle swarm optimization” by Mojtaba Ahmadieh Khanesar, Mohammad Teshnehlab, Mahdi Aliyari Shoorehdeli (2007 Mediterranean Conference on Control & Automation, Athens, 2007, pp. 1-6. doi: 10.1109/MED.2007.4433821). MBPSO is an efficient BPSO algorithm. See H. Nezamabadi-pour, M. Rostami-shahrbabaki, M. M. Farsangi, “Binary Particle Swarm Optimization: challenges and New Solutions”, The Journal of Computer Society of Iran (CSI) On Computer Science and Engineering (JCSE), vol. 6, no. (1-A), pp. 21-32, 2008. However, MBPSO only applies when optimizing a single objective. In contrast, MOMBPSO, as described herein, combines MBPSO with particle swarm optimization algorithms for optimizing multiple objectives. See Torres et al, “Particle swarm optimization algorithms for solving many-objective problems,” 2015 J. Comp. Int. Sci. 6(2):61-70, available at http://epacis.net/jcis/PDF_JCIS/JCIS-0095.pdf.

Specifically, MOMBPSO iteratively analyzes a plurality of particles to generate a collection of optimal solutions that balance tradeoffs between the multiple objectives. These solutions can be arranged onto a two-dimensional plot as a Pareto front, as described in detail below. Although the systems and methods described herein utilize two objectives, those of skill in the art will appreciate that more than two objectives may be used with MOMBPSO.

To generate the collection of solutions, a plurality of inputs is provided to the MOMBPSO, The following Table 1 lists a plurality of example inputs and example values for those inputs:

TABLE 1

| Parameter | Value | Description |
|---|---|---|
| Max inertia | 0.9 | Maximum inertia of the particles, |
| | | inertia |
| Min inertia | 0.4 | decreases from Max to Min inertia |
| | | linearly throughout the generations |
| Cognitive velocity | 2 | c1 term |
| Social velocity | 2 | c2 term |
| Max velocity | 4 | Constrained maximum velocity of the |
| | | particles |
| Number of particles | 10-20 | Number of particles in the population |
| Max generation | 100 | Maximum number of iterations for the |
| | | algorithm, can be a termination criterion |

Those of skill in the art will appreciate that other inputs and/or other values for the inputs listed above may be used. Notably, modifying the inputs will result in MOMBPSO generating a different collection of solutions.

In the embodiments described herein, the two objectives targeted by the MOMBPSO are i) maximizing DBS therapy efficiency and ii) reducing stimulation power utilization. In the systems and methods described herein, DBS therapy efficiency is quantified using an error index that represents thalamic (TH) neuron firing fidelity to the sensorimotor cortex (SMC) input. Specifically, as defined herein, error index is equal to a total number of errors divided by a total number of SMC inputs. The lower the error index, the better the DBS therapy efficiency.

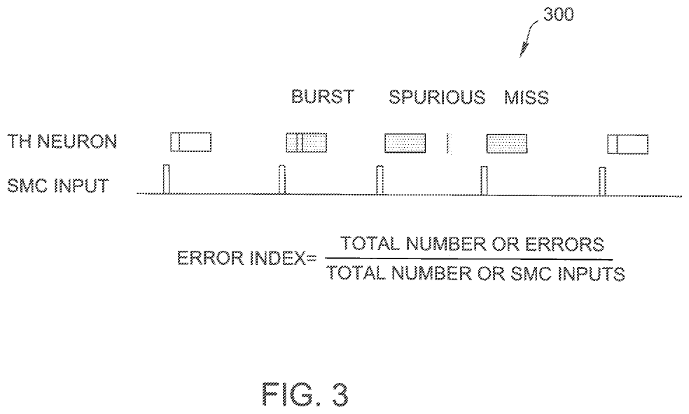

FIG. 3 is a schematic diagram 300 illustrating the error index. In diagram 300, a first SMC input results in a proper TH neuron firing, a second SMC input results in a burst TH neuron firing (i.e., an error), a third SMC input results in a spurious TH neuron firing (i.e., an error), a fourth SMC input results in a missed TH neuron firing (i.e., an error), and a fifth SMC input results in a proper TH neuron firing. Accordingly, the error index is equal to ⅗, or 0.6.

In the embodiments described herein, power utilization is quantized as the instantaneous stimulation frequency (i.e., the number of stimulation pulses delivered per second). Accordingly, the Pareto front generated using the systems and methods described herein has two dimensions: error index versus instantaneous stimulation frequency.

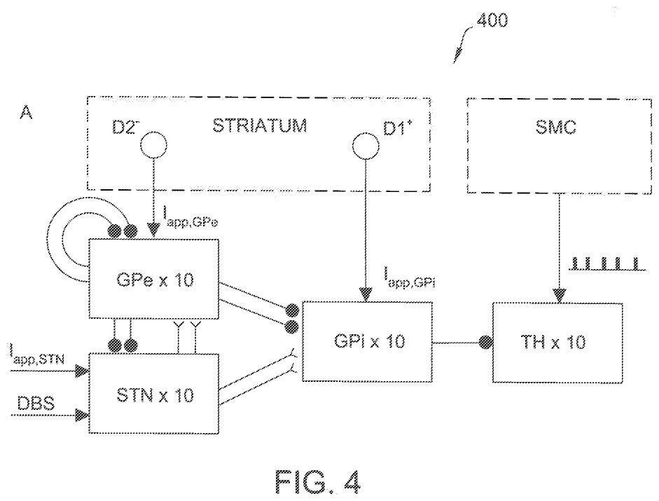

As noted above, in the systems and methods described herein, MOMBPSO is applied to the RT model. FIG. 4 is a schematic diagram of the RT model 400. RT model 400 represents the basal ganglia. In the embodiments described herein, a stimulation pattern is represented as a fixed-length binary segment with a 1 millisecond (ms) resolution. A binary value of 1 means that there is a stimulation pulse at the corresponding 1 ms bin, and a binary value of 0 means that there is no stimulation pulse at the corresponding bin. They length of the binary segment may be varied, such as 100, 200, or 400 ms segments. To create a full stimulation pulse train, the binary segments are repeated for a total stimulation time, and are input into the cells in the stimulated nucleus (e.g., the STN or the GPi) in RT model 400.

As shown in FIG. 4, RT model 400 includes the GPe, the STN, the GPi, the thalamus (TH), and an input action potential train from the SMC at 14 Hz (±20%). Applied currents (Iₐₚₚ) representing inputs to the GPe, GPi, and STN are modeled. The GPe primarily receives inputs from striatal neurons expressing inhibitory D2-type receptors, while the GPi primarily receives inputs from striatal neurons expressing excitatory D1-type receptors. Excitatory and inhibitory synapses are depicted using forked and circular terminations, respectively. The RT model is described in further detail in “High frequency stimulation of the subthalamic nucleus eliminates pathological thalamic rhythmicity in a computational model” by Rubin, J. E. & Terman, D. J. (Camput Neurosci (2004) 16: 211, available at https://doi.org/10.1023/B:JCNS.0000025686.47117.67) and Science Translational Medicine 4 Jan 2017: Vol. 9, Issue 371, eaah3532 DOI: 10.1126/scitranslmed.aah3532.

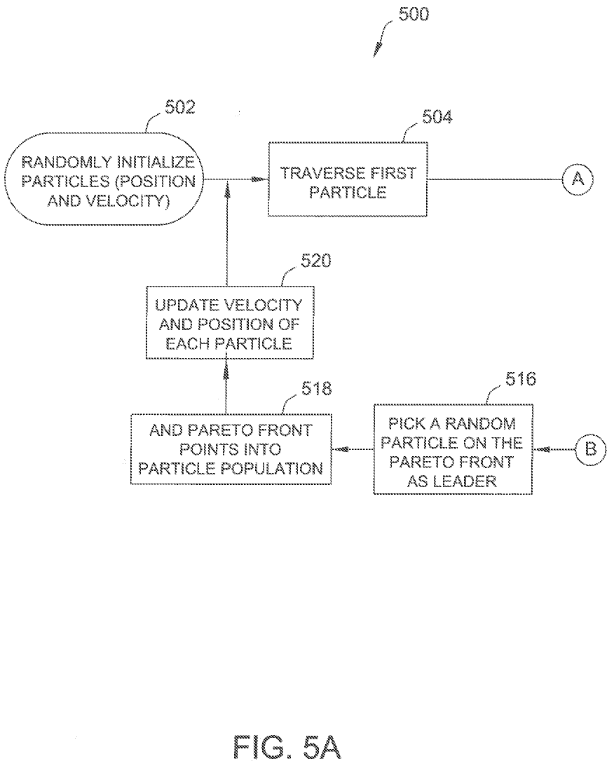

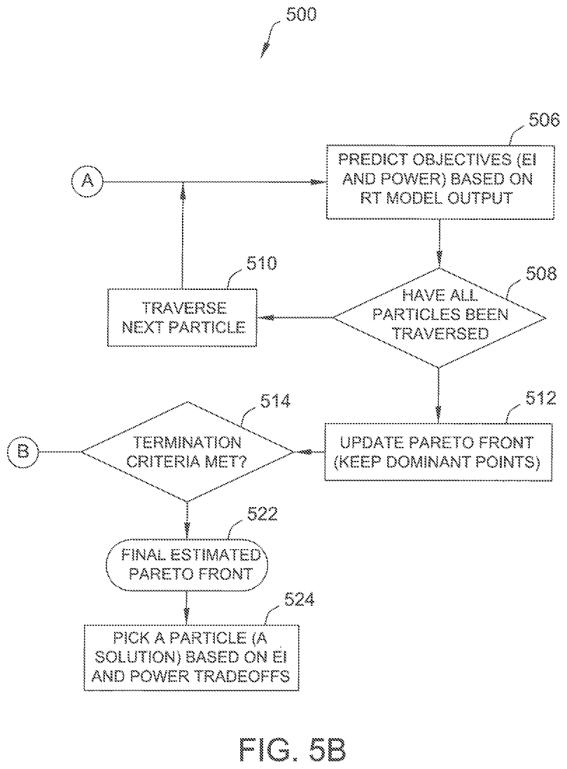

FIGS. 5A and 5B are a flow diagram of one embodiment of a method 500 for generating DBS stimulation patterns using MOMBPSO. Method 500 may be implemented, for example, using computing device 200 (shown in FIG. 2). Method 500 is an iterative process. For each iteration (referred to as a generation), all particles are traversed and evaluated using the RT model. After each generation, the most optimal solutions are evaluated with a Pareto dominance method (i.e., improving at least one objective better without negatively impacting any other objective) and collected into an archive referred to as a Pareto front. A random leader on the Pareto front is picked, and the Pareto front points are added back into the population of particles with their velocities and positions updated.

The iterative process ends when a predetermined termination criterion is reached (e.g., a predetermined number of generations), and a final Pareto front is generated. Method 500 will now be described in detail.

To begin method 500, a plurality of particles is randomly initialized with respective positions and velocities at block 502. In some embodiments, this process may be further assisted by incorporating a priori knowledge of known stimulation patterns (such as 100 or 130 Hz tonic stimulation) to expedite the search process. Row proceeds to block 504, at which a first particle of the plurality of particles is traversed. Flow then proceeds to block 506, where the objectives (e.g., the error index and the instantaneous stimulation frequency) are predicted based on the output of the RT model. Flow then proceeds to block 508.

At block 508, if not all particles have been traversed, flow proceeds to block 510, and the next particle is traversed before the flow returns to block 506. Once all particles have been traversed, flow proceeds from block 508 to block 512. Reaching block 512 represents completing a generation of the iteration. At block 512, the Pareto front is updated, keeping the dominant points using the Pareto dominance method. Flow then proceeds to block 514.

At block 514, it is determined whether a predetermined termination criterion has been met. In one example, the predetermined termination criterion constitutes completing a certain number of generations (e.g., 100 generations). Alternatively, the predetermined termination criterion may be any suitable criterion.

If the termination criteria have not been met, flow proceeds to block 516, and a random particle on the Pareto front is selected as a leader. The leader is used to update the social velocity (listed in Table 1 above). Flow then proceeds to block 518, where all the Pareto front points are added into the particle population, and then proceeds to block 520, where the velocity and position of each particle are updated. From block 520, flow returns to block 504 to start a new generation.

If the termination criteria are met, flow proceeds from block 514 to block 522, and a final estimated Pareto front is generated. Then, at block 524, a particle on the final estimated Pareto front can be selected as a solution, based on tradeoffs between the error index and instantaneous stimulation frequency. The solution particle may be selected by a user (e.g., in response to computing device 200 presenting the final estimated Pareto front to the user), or may be automatically selected by computing device 200.

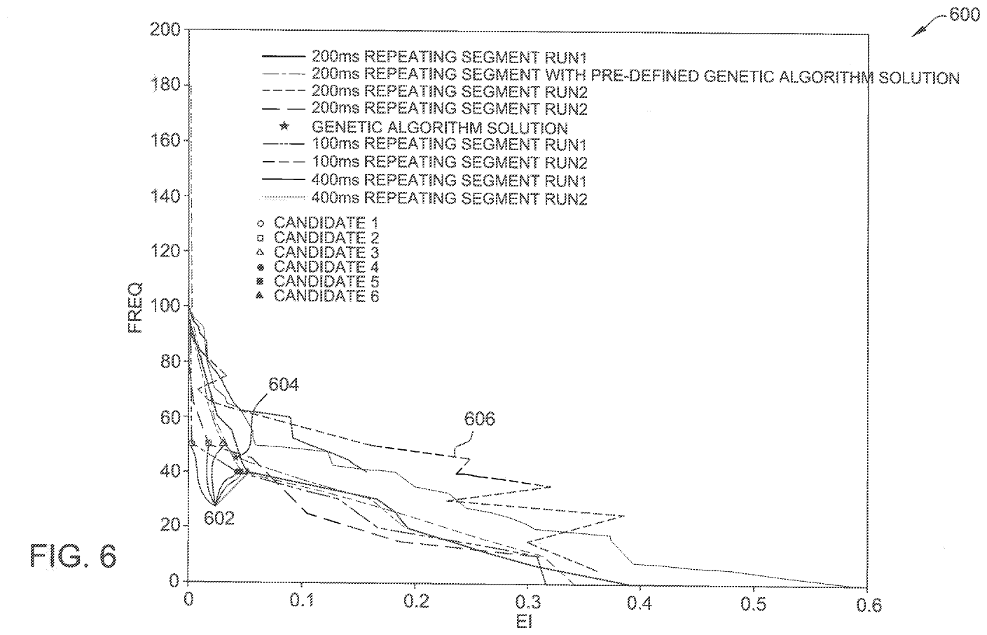

FIG. 6 is a graph 600 showing multiple final estimated Pareto fronts generated using method 500 (shown in FIGS. 5A and 5B). As shown in FIG. 6, graph 600 includes a solution particle 602 (representing a candidate stimulation pattern) for each Pareto front. As noted above, in the embodiments described herein, the Pareto front plots error index versus instantaneous stimulation frequency, where lower values of error index and instantaneous stimulation frequency are desirable. Accordingly, the solution particle 602 for each Pareto front is selected based on desired tradeoffs of DBS therapy efficacy and power utilization,

In graph 600, Pareto fronts are generated over multiple runs at various repeating segments (e.g., 100 ms, 200 ms, and 400 ms). For comparison, a genetic algorithm solution 604 and a tonic stimulation pattern 606 are also indicated on graph 600. As demonstrated by graph 600, the Pareto fronts generated with 100 and 200 ms repeating segments showed similar or better performance than genetic algorithm solution 604, and better performance than tonic stimulation pattern 606.

The following Table 2 lists the final stimulation pattern candidates identified from graph 600:

TABLE 2

| | | | | Pattern, as given by |
|---|---|---|---|---|
| | Error Index | Inst. | Segment | time stamps of the |
| Candidate | (EI) | Freq. | length | pulse onset (ms) |
| 1 | 0.003 | 50 Hz | 100 ms | [24, 28, 54, 71, 76] |
| 2 | 0.017 | 50 Hz | 200 ms | [6, 11, 32, 54, 59, 103, 107, 157, 164, 178, 193] |
| 3 | 0.029 | 50 Hz | 200 ms | [47, 52, 63, 68, 72, 124, 138, 143, 193, 199] |
| 4 | 0.042 | 40 Hz | 100 ms | [24, 28, 71, 76] |
| 5 | 0.045 | 40 Hz | 100 ms | [40, 44, 76, 81] |
| 6 | 0.050 | 40 Hz | 200 ms | [10, 12, 36, 70, 98, 105, 181, 195] |

As shown in the last column of Table 2, the stimulation pattern is defined by a series of time stamps. For example, for the first candidate, five pulses occur over a repeating 100 ms segment. The first pulse occurs at 24 ms, the second pulse occurs at 26 ms, the third pulse occurs at 54 ms, the fourth pulse occurs at 71 ms, and the fifth pulse occurs at 76 ms.

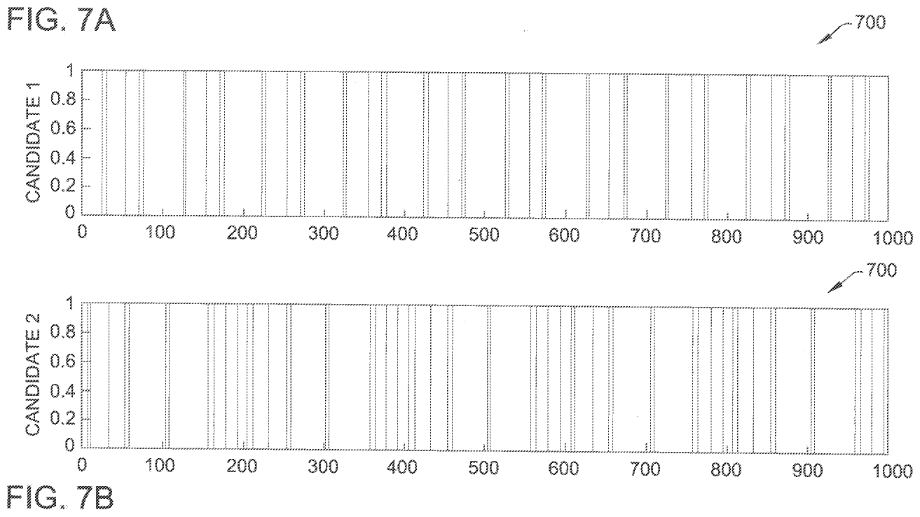

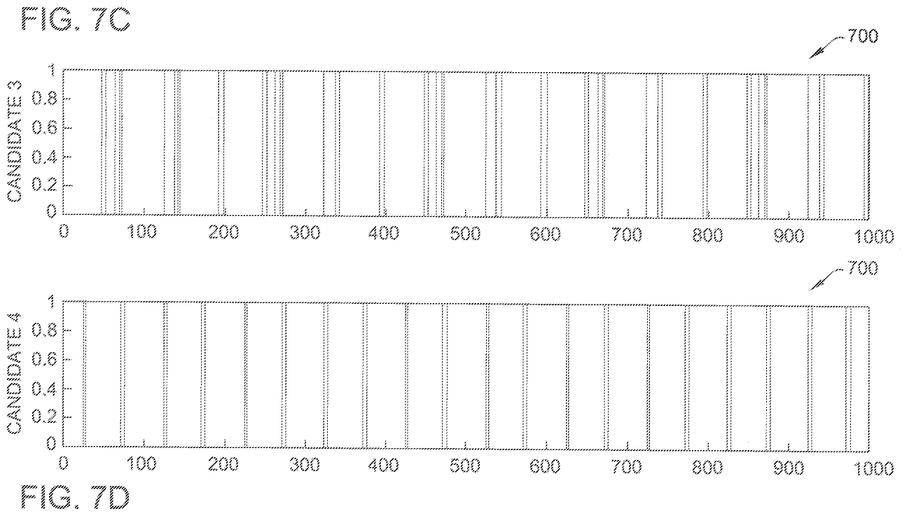

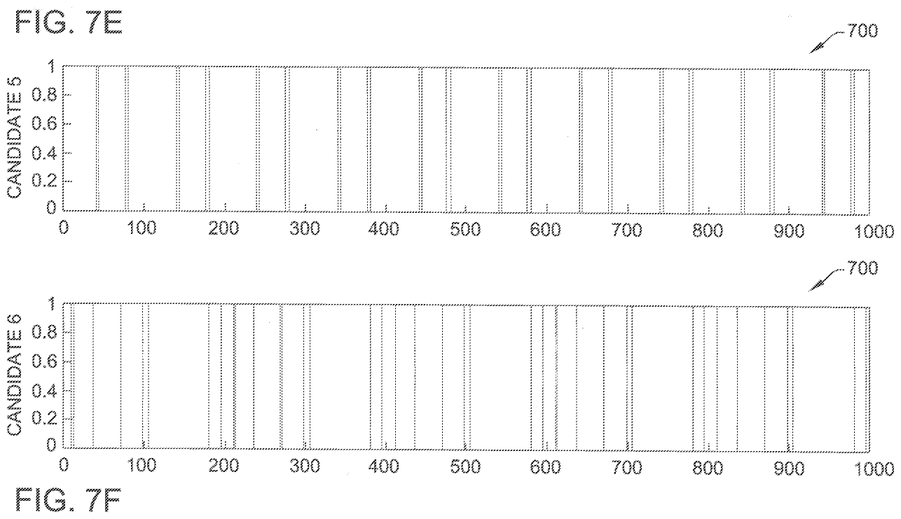

FIGS. 7A-7F are graphs 700 showing the six candidate stimulation patterns listed in Table 2. In graphs 700, the x-axis is time (in ms), and the y-axis is binary (i.e., 0 or 1, with values of 1 where pulses occur). Those of skill in the art will appreciate that when delivering actual pulses, pulse waveform shape and amplitude may be chosen and scaled according to user specifications.

Those of skill in the art will appreciate that modifications may be made to the above embodiments without departing from the spirit and scope of the disclosure. For example, in one embodiment, an alternative computational circuit model (i.e., other than the RT model) may be used to identify optimal stimulation patterns for DBS in a different brain target (e.g., the ventral intermediate (VIM) nucleus of the TH).

In another embodiment, the systems and methods described herein could be used to identify optimal stimulation patterns for spinal cord stimulation (SCS) using a spinal cord circuitry model, or to identify optimal stimulation patterns for peripheral nerve stimulation using a suitable model.

The stimulation patterns generated using the system and methods described herein may be used to replace or supplement current waveform patterns (e.g., 130 Hz tonic stimulation) in existing stimulation devices (e.g., primary-cell implantable pulse generators). This may be advantageous, as the stimulation patterns generated using MOMBPSO may stimulate at approximately 40-50 Hz, resulting in a reduction in power utilization of 61-70%.

In some embodiments, other optimization objectives (i.e., other than therapy efficacy and power utilization) may be used by the MOMBPSO. These other objectives may be identified, for example, by analyzing patient-specific or population data such as lead localization, local field potential records, etc., which may allow for better efficacy of DBS therapy. In yet other embodiments, the MOMBPSO algorithm may be implemented in a closed-loop fashion using a cloud-computing platform, allowing for real-time feedback to be delivered to clinicians. This functionality could also be implemented in clinician programmers.

The embodiments described herein provide systems and methods for generating stimulation patterns (e,g., for DBS) using MOMBPSO. Using MOMBPSO, multiple objectives (e.g., DBS therapy efficiency and power utilization) of optimization are achievable. Further, MOMBSPSO may be coupled with the Rubin-Terman model of the basal ganglia to generate new, improved stimulation patterns. For example, the systems and methods described herein may be used to generate stimulation patterns that are as effective as 100+ Hz tonic stimulation, but with lower power utilization, or stimulation patterns that are more effective than 100+ Hz tonic stimulation, with equivalent power utilization.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of dements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A computing device for generating stimulation patterns for neurostimulation, the computing device comprising:
a processor; and
a memory device communicatively coupled to the processor, the memory device including instructions that, when executed, cause the processor to:
provide a plurality of inputs to a multi-objective modified binary particle swarm optimization (MOMBPSO) algorithm; and
apply the MOMBPSO to a computational circuit model using the plurality of inputs to generate a plurality of candidate stimulation patterns, wherein the MOMBPSO is applied to the computational circuit model to optimize both i) therapy efficacy and ii) power utilization.

2. The computing device of claim 1, wherein to apply the MOMBPSO, the instructions cause the processor to apply the MOMBPSO to the Rubin-Terman model to generate a plurality of candidate stimulation patterns for deep brain stimulation (DBS).

3. The computing device of claim 2, wherein therapy efficacy is quantized as an error index, the error index defined as a total number of errors divided by a total number of sensorimotor cortex (SMC) inputs.

4. The computing device of claim 1, wherein power utilization is quantized as a number of stimulation pulses delivered per second.

5. The computing device of claim 1, wherein the instructions further cause the processor to generate at least one Pareto front including the plurality of candidate stimulation patterns.

6. The computing device of claim 1, wherein the MOMBPSO is applied to the computational circuit model to maximize therapy efficacy and minimize power utilization.

7. The computing device of claim 1, wherein the computing device is implemented within an implantable pulse generator.

8. The computing device of claim 1, wherein the instructions cause the processor to apply the MOMBPSO to the computational circuit model to optimize at least one objective in addition to therapy efficacy and power utilization.

9. The computing device of claim 1, wherein the instructions cause the processor to apply the MOMBPSO to generate stimulation patterns that target one of the subthalamic nucleus, the globus pallidus interna, the globus pallidus externa, and the ventral intermediate nucleus of the thalamus.

10. The computing device of claim 1, wherein to apply the MOMBPSO, the instructions cause the processor to apply the MOMBPSO to generate a plurality of candidate stimulation patterns for one of spinal cord stimulation and peripheral nerve stimulation.

11. A computer-implemented method of generating stimulation patterns for neurostimulation, the method comprising:
providing, using a processor, a plurality of inputs to a multi-objective modified binary particle swarm optimization (MOMBPSO) algorithm; and
applying, using the processor, the MOMBPSO to a computational circuit model using the plurality of inputs to generate a plurality of candidate stimulation patterns, wherein the MOMBPSO is applied to the computational circuit model to optimize both i) therapy efficacy and ii) power utilization.

12. The method of claim 11, further comprising applying one of the plurality of candidate stimulation patterns to a patient using a stimulation system.

13. The method of claim 11, wherein applying the MOMBPSO comprises applying MOMBPSO to the Rubin-Terman model to generate a plurality of candidate stimulation patterns for deep brain stimulation (DBS).

14. The method of claim 13, wherein therapy efficacy is quantized as an error index, the error index defined as a total number of errors divided by a total number of sensorimotor cortex (SMC) inputs.

15. The method of claim 11, wherein power utilization is quantized as a number of stimulation pulses delivered per second.

16. The method of claim 11, further comprising generating at least one Pareto front including the plurality of candidate stimulation patterns.

17. The method of claim 11, wherein the processor is implemented within an implantable pulse generator.

18. The method of claim 11, wherein applying the MOMBPSO comprises applying the MOMBPSO to the computational circuit model to optimize at least one objective in addition to therapy efficacy and power utilization.

19. The method of claim 11, wherein applying the MOMBPSO comprises applying the MOMBPSO to generate stimulation patterns that target one of the subthalamic nucleus, the globus pallidus interna, the globus pallidus externa, and the ventral intermediate nucleus of the thalamus.

20. The method of claim 11, wherein applying the MOMBPSO comprises applying the MOMBPSO to generate a plurality of candidate stimulation patterns for one of spinal cord stimulation and peripheral nerve stimulation.
